(19) United States
(12) Patent Application Publication
(10) Pub. No.: US 2025/0029693 A1
(43) Pub. Date: Jan. 23, 2025

(54) INFORMATION PROCESSING APPARATUS, INFORMATION PROCESSING METHOD, AND INFORMATION PROCESSING PROGRAM

(71) Applicant: FUJIFILM CORPORATION, Tokyo (JP)

(72) Inventor: Yuki WADA, Tokyo (JP)

(21) Appl. No.: 18/907,594

(22) Filed: Oct. 7, 2024

Related U.S. Application Data
(63) Continuation of application No. PCT/JP2023/013731, filed on Mar. 31, 2023.

(30) Foreign Application Priority Data
Apr. 18, 2022 (JP) 2022-068442

(51) Int. Cl. G16H 10/60

(52) U.S. Cl. CPC G16H 10/60

(57) ABSTRACT

An information processing apparatus includes at least one processor. The processor is configured to acquire a medical document to which medical department information indicating a medical department that is a creation source and date information indicating a creation date are assigned, and generate list information in which information on a corresponding medical document is recorded in a format in which an identification name of the medical department is taken on a first axis and a date is taken on a second axis intersecting the first axis.

### CROSS-REFERENCE TO RELATED APPLICATIONS

This application is a continuation application of International Application No. PCT/JP2023/013731, filed Mar. 31, 2023, the disclosure of which is incorporated herein by reference in its entirety. Further, this application claims priority from Japanese Patent Application No. 2022-068442, filed on Apr. 18, 2022, the disclosure of which is incorporated herein by reference in its entirety.

### BACKGROUND

### 1. Technical Field

The technology of the present disclosure relates to an information processing apparatus, an information processing method, and an information processing program.

### 2. Description of the Related Art

As a technology for facilitating understanding of a treatment status of a patient, the following technology is known. For example, JP2020-4244A discloses a medical support device comprising a display screen generation unit that generates a display screen on which patient identification information and a medical treatment process are displayed in association with each other for each patient for a plurality of patients, and an unread management unit that displays a status indicating that the medical treatment process is unread, read, or both of these pieces of information on the display screen. The display screen generation unit generates a timeline display screen on which the medical treatment process is displayed in time series for a specific one patient, and the unread management unit displays the status of the medical treatment process on the timeline display screen.

### SUMMARY

Medical documents, such as an interpretation report and an electronic medical record, are created or updated daily. In a case in which the information described in these medical documents can be confirmed from an overarching perspective, a status of the patient can be understood from an overarching perspective, which is convenient. In particular, in a case in which the patient undergoes the medical treatment from a plurality of medical departments, a mechanism for confirming the information for each medical department from an overarching perspective is desired.

An object of the technology of the present disclosure is to enable understanding of information on a medical document created in a plurality of medical departments from an overarching perspective.

The technology of the present disclosure relates to an information processing apparatus comprising at least one processor. The processor is configured to acquire a medical document to which medical department information indicating a medical department that is a creation source and date information indicating a creation date are assigned. The processor is configured to generate list information in which information on a corresponding medical document is recorded in a format in which an identification name of the medical department is taken on a first axis and a date is taken on a second axis intersecting the first axis.

According to the technology of the present disclosure, it is possible to understand information on a medical document created in a plurality of medical departments from an overarching perspective.

### BRIEF DESCRIPTION OF THE DRAWINGS

Exemplary embodiments according to the technique of the present disclosure will be described in detail based on the following figures, wherein:

FIG. 1 is a diagram showing an example of a configuration of a medical information management system according to an embodiment of the technology of the present disclosure;

FIG. 2 is a diagram showing an example of a hardware configuration of an information processing server according to the embodiment of the technology of the present disclosure;

FIG. 3 is a functional block diagram showing an example of a functional configuration of the information processing server according to the embodiment of the technology of the present disclosure;

FIG. 4 is a diagram showing an example of list information according to the embodiment of the technology of the present disclosure;

FIG. 5 is a flowchart showing an example of a flow of processing performed by executing an information processing program via a CPU according to the embodiment of the technology of the present disclosure;

FIG. 6 is a functional block diagram showing an example of the functional configuration of the information processing server according to the embodiment of the technology of the present disclosure;

FIG. 7 is a flowchart showing an example of a flow of processing performed by executing an information processing program via a CPU according to the embodiment of the technology of the present disclosure;

FIG. 8 is a flowchart showing an example of a flow of the processing performed by executing the information processing program via the CPU according to the embodiment of the technology of the present disclosure; and

FIG. 9 is a diagram showing an example of the list information according to the embodiment of the technology of the present disclosure.

### DETAILED DESCRIPTION

Hereinafter, examples of embodiments of the present invention will be described with reference to the accompanying drawings. It should be noted that the same or equivalent components and parts in the respective drawings are denoted by the same reference numerals, and the duplicated description will be omitted.

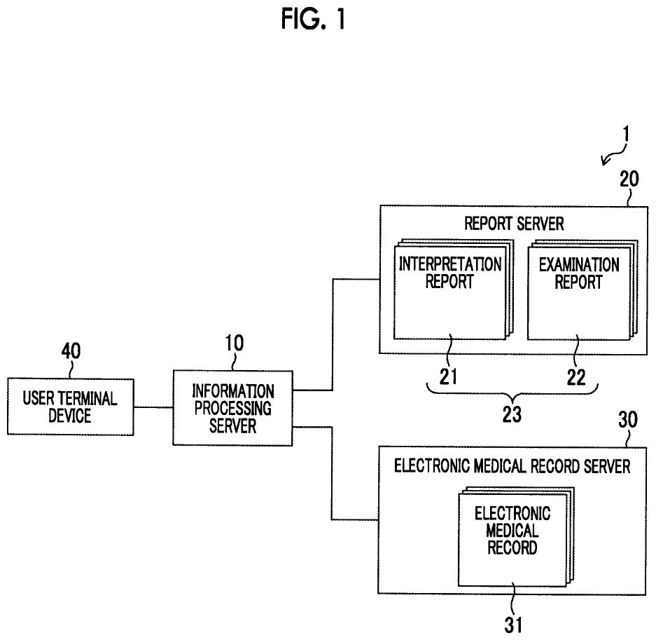

FIG. 1 is a diagram showing an example of a configuration of a medical information management system 1 according to the embodiment of the technology of the present disclosure. The medical information management system 1 has a configuration including an information processing server 10, a report server 20, an electronic medical record server 30, and a user terminal device 40.

The report server 20 is a server computer comprising a storage medium that stores medical documents such as an interpretation report 21 and an examination report 22. In the interpretation report 21, for example, information on a disease extracted from medical images, such as a plain X-ray image, a CT image, a mammography image, or an MRI image, is described. In the examination report 22, for example, information on a result of an examination performed on a tissue or an organ collected from a patient is described. The interpretation report 21 and the examination report 22 are created for each patient. A plurality of interpretation reports 21 and a plurality of examination reports 22 may be created for one patient. In addition, the interpretation report 21 and the examination report 22 may be created for one patient in a plurality of different medical departments. The interpretation report 21 and the examination report 22 are created for each medical department.

Patient identification (ID) for identifying the patient, medical department information for identifying the medical department that creates the report, and date information indicating the date on which the report is created are assigned to each of the interpretation report 21 and the examination report 22. It should be noted that, in a case in which the distinction between the interpretation report 21 and the examination report 22 is not made or the distinction between the interpretation report 21 are collectively referred to, the interpretation report 21 and the examination report 22 are simply referred to as “report 23” in the following description. The report 23 is an example of a “medical document” according to the technology of the present disclosure.

The electronic medical record server 30 is a server computer comprising a storage medium that stores the electronic medical record 31 created for each patient. The electronic medical record 31 includes information on a treatment for a disease of the patient or course information on the disease of the patient. Examples of the “information on the treatment” include subjective information, objective information, assessment, plan, a medication status, an examination result, an examination order, and the like for the disease.

A doctor who creates the electronic medical record 31 registers the disease described in the report 23 as a disease that needs to be treated for the patient by using a registration system (not shown). The registered disease is recorded in the electronic medical record 31 through system linkage. The electronic medical record 31 is updated daily. The electronic medical record 31 may be created for one patient in a plurality of different medical departments. The electronic medical record 31 is created for each medical department.

Patient identification (ID) for identifying the patient, medical department information for identifying the medical department that creates the electronic medical record 31, and date information indicating the date on which the electronic medical record 31 is created are assigned to the electronic medical record 31. The electronic medical record 31 is an example of a “medical document” according to the technology of the present disclosure.

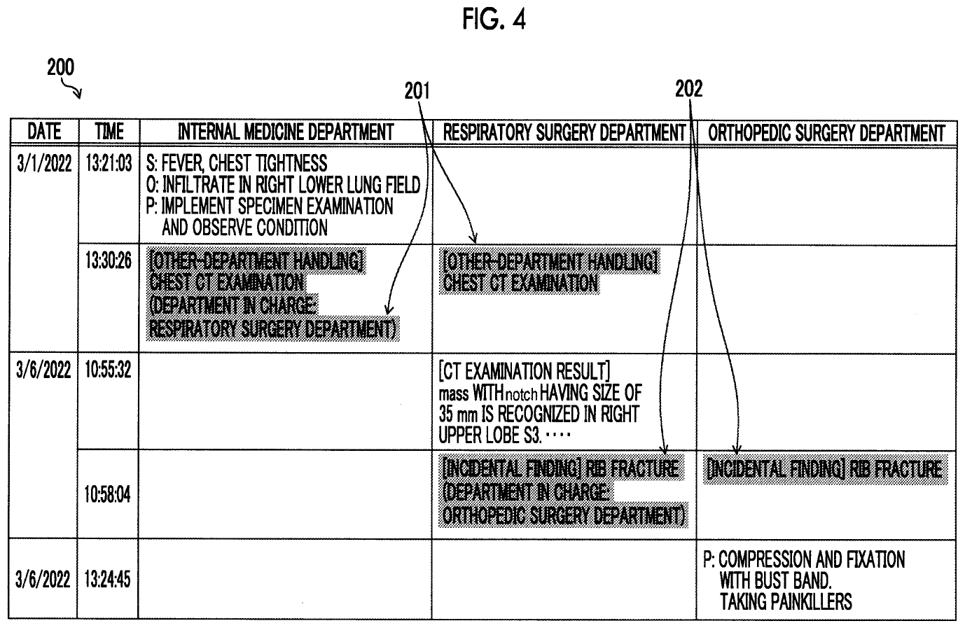

The information processing server 10 generates list information 200 (see FIG. 4) in which the information on the report 23 and the electronic medical record 31 is aggregated for a specific patient designated by the user terminal device 40. Details of the list information 200 will be described later. The information processing server 10 is an example of an “information processing apparatus” according to the technology of the present disclosure.

The user terminal device 40 is a computer intended to be used by a doctor. The user terminal device 40 requests the information processing server 10 to present the list information 200 for the specific patient. The patient who is a target is designated by the user terminal device 40. The information processing server 10 generates the list information 200 for the designated patient in response to the request from the user terminal device 40, and transmits the generated list information 200 to the user terminal device 40. A display (not shown) of the user terminal device 40 that has received the list information 200 displays the list information 200 transmitted from the information processing server 10.

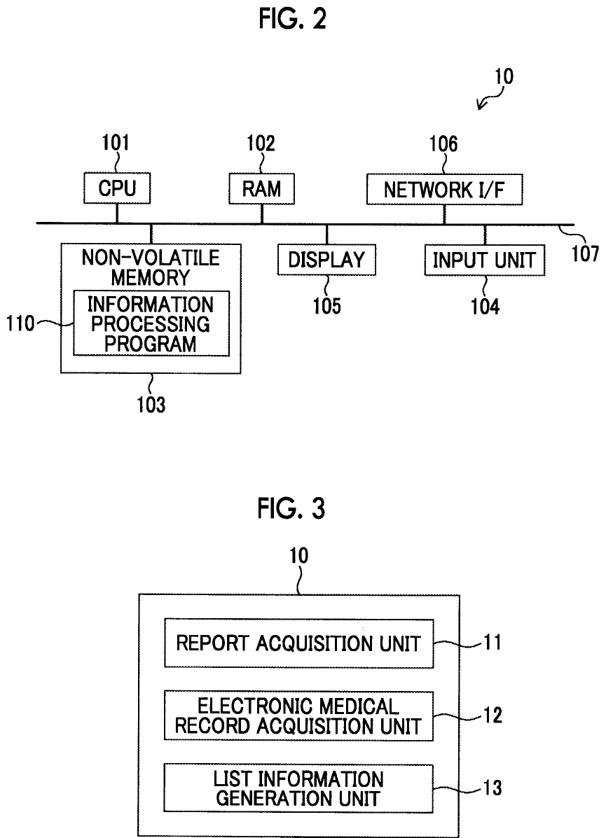

FIG. 2 is a diagram showing an example of a hardware configuration of the information processing server 10. The information processing server 10 includes a central processing unit (CPU) 101, a random access memory (RAM) 102, a non-volatile memory 103, an input device 104, a display 105, and a network interface 106. These pieces of hardware are connected to a bus 107.

The display 105 is, for example, a liquid crystal display or a light emitting diode (LED) display. The input device 104 includes, for example, a keyboard and a mouse, and may include a proximity input device such as a touch panel display, a voice input device such as a microphone, and a contactless input device such as a camera or a sensor. The network interface 106 is an interface for connecting the information processing server 10 to the report server 20, the electronic medical record server 30, and the user terminal device 40 via a network.

The non-volatile memory 103 is a non-volatile storage medium such as a hard disk and a flash memory. The non-volatile memory 103 stores an information processing program 110. The RAM 102 is a work memory for the CPU 101 to execute processing. The CPU 101 loads the information processing program 110 stored in the non-volatile memory 103 into the RAM 102, and executes the processing according to the information processing program 110. The CPU 101 is an example of a “processor” according to the technology of the present disclosure.

FIG. 3 is a functional block diagram showing an example of a functional configuration of the information processing server 10. The information processing server 10 includes a report acquisition unit 11, an electronic medical record acquisition unit 12, and a list information generation unit 13. The CPU 101 executes the information processing program 110, whereby the CPU 101 functions as the report acquisition unit 11, the electronic medical record acquisition unit 12, and the list information generation unit 13.

The report acquisition unit 11 accesses the report server 20 to acquire the report 23 related to the specific patient from among the reports 23 stored in the storage medium provided in the report server 20. The specific patient is designated by the user terminal device 40. The patient ID is assigned to the interpretation report 21 and the examination report 22. The report acquisition unit 11 transmits a report acquisition request, including the patient ID, to the report server 20. The report server 20 extracts the report to which the patient ID related to the acquisition request is assigned, from among the reports 23 stored in the storage medium provided in the report server 20, and transmits the extracted report to the information processing server 10. The report acquisition unit 11 acquires the report 23 transmitted from the report server 20. It should be noted that, in a case in which the report 23 is created for one patient in the plurality of different medical departments, all of the reports 23 created in the plurality of respective medical departments are acquired.

The report server 20 may comprise a report database (not shown) in which the patient ID and identification information (report ID) of the interpretation report 21 and the examination report 22 are associated with each other. In such a case, the report server 20 may extract the report 23 related to the specific patient by using the report database.

The electronic medical record acquisition unit 12 accesses the electronic medical record server 30 to acquire the electronic medical record 31 related to the specific patient from among the electronic medical records 31 stored in the storage medium provided in the electronic medical record server 30. The patient ID is assigned to the electronic medical record 31. The electronic medical record acquisition unit 12 transmits an electronic medical record acquisition request, including the patient ID, to the electronic medical record server 30. The electronic medical record server 30 extracts the electronic medical record 31 to which the patient ID related to the acquisition request is assigned, from among the electronic medical records 31 stored in the storage medium provided in the electronic medical record server 30, and transmits the extracted electronic medical record 31 to the information processing server 10. The electronic medical record acquisition unit 12 acquires the electronic medical record 31 transmitted from the electronic medical record server 30. It should be noted that, in a case in which the electronic medical record 31 is created for one patient in the plurality of medical departments, all of the electronic medical records 31 created in the plurality of respective medical departments are acquired.

The electronic medical record server 30 may comprise an electronic medical record database (not shown) in which the patient ID and the identification information (electronic medical record ID) of the electronic medical record 31 are associated with each other. In such a case, the electronic medical record server 30 may extract the electronic medical record 31 related to the specific patient by using the electronic medical record database.

The list information generation unit 13 generates the list information 200 that is edited such that the information described in the report 23 acquired by the report acquisition unit 11 and the electronic medical record 31 acquired by the electronic medical record acquisition unit 12 can be confirmed from an overarching perspective.

FIG. 4 is a diagram showing an example of the list information 200 generated by the list information generation unit 13. In the list information 200, information is recorded in which the information described in the corresponding report 23 or electronic medical record 31 is described in a matrix format in which an identification name of the medical department is taken on a first axis (horizontal axis) and a date is taken on a second axis (vertical axis) intersecting the first axis.

The list information generation unit 13 specifies, based on the medical department information and the date information assigned to the report 23 acquired by the report acquisition unit 11 and the electronic medical record 31 acquired by the electronic medical record acquisition unit 12, a recording column of the information described in the medical documents within the format, and records the information corresponding to the specified recording column. As a result, the list information 200 in which the information described in the medical document created for the specific patient in each of the plurality of medical departments is presented in time series for each medical department is generated. In the list information 200 shown in FIG. 4, the information described in the report 23 or the electronic medical record 31 created in an internal medicine department, a respiratory surgery department, and an orthopedic surgery department is presented in time series for each medical department. The user may be able to designate the medical department for which the information is presented in the list information 200. In such a case, the list information generation unit 13 generates the list information 200 in which the information is recorded in a format in which the identification name of the designated medical department is taken on the first axis.

In the present embodiment, the list information generation unit 13 generates a summary of the information described in the report 23 or the electronic medical record 31, to record the generated summary in a corresponding recording column of the format. The information can be summarized by using an existing natural language analysis technique. The list information generation unit 13 transmits the generated list information 200 to the user terminal device 40. The display of the user terminal device 40 that has received the list information 200 displays the list information 200.

Since the information presented in the list information 200 is the summary of the information described in the report 23 or the electronic medical record 31, the user may want to confirm the details of the information described in the report 23 or the electronic medical record 31. The list information generation unit 13 presents the details of the information described in the report 23 or the electronic medical record 31 in response to a request from the user terminal device 40. The user can, for example, click any cell, in which the summarized information is described, in the list information 200 displayed on the display of the user terminal device 40, to request the details of the information described in the cell to the information processing server 100. The information processing server 100 that has received the request transmits the details (detailed information) of the information described in the report 23 or the electronic medical record 31 in response to the request to the user terminal device 40. The detailed information may be, for example, the original text of the text described in the report 23 or the electronic medical record 31. The display of the user terminal device 40 displays the detailed information transmitted from the information processing server 100. The detailed information may be displayed in a form of a mouse-over pop-up along with the list information 200.

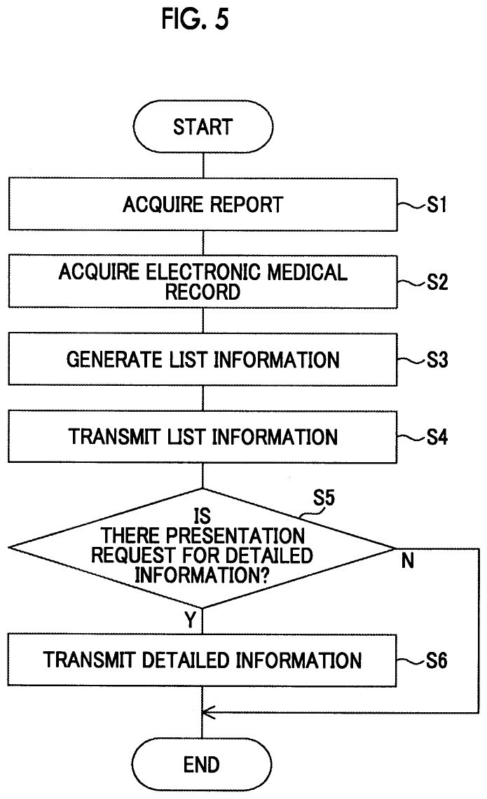

FIG. 5 is a flowchart showing an example of a flow of processing performed by executing the information processing program 110 via the CPU 101 according to the embodiment of the technology of the present disclosure. The information processing program 110 is executed, for example, in a case in which a presentation request for the list information 200 is issued from the user terminal device 40.

In step S1, the report acquisition unit 11 accesses the report server 20 to acquire the report 23 related to the specific patient from among the reports 23 stored in the storage medium provided in the report server 20. Specifically, the report acquisition unit 11 transmits the report acquisition request, including the patient ID, to the report server 20. The report server 20 extracts the report to which the patient ID related to the acquisition request is assigned, from among the reports 23 stored in the storage medium provided in the report server 20, and transmits the extracted report to the information processing server 10. The report acquisition unit 11 acquires the report 23 transmitted from the report server 20. In a case in which the report 23 is created for the patient related to the acquisition request in the plurality of different medical departments, all of the reports 23 created for the patient in the plurality of respective medical departments are acquired.

In step S2, the electronic medical record acquisition unit 12 accesses the electronic medical record server 30 to acquire the electronic medical record 31 related to the specific patient from among the electronic medical records 31 stored in the storage medium provided in the electronic medical record server 30. Specifically, the electronic medical record acquisition unit 12 transmits the electronic medical record acquisition request, including the patient ID, to the electronic medical record server 30. The electronic medical record server 30 extracts the electronic medical record to which the patient ID related to the acquisition request is assigned, from among the electronic medical records 31 stored in the storage medium provided in the electronic medical record server 30, and transmits the extracted electronic medical record 31 to the information processing server 10. The electronic medical record acquisition unit 12 acquires the electronic medical record 31 transmitted from the electronic medical record server 30. In a case in which the electronic medical record 31 is created for the patient related to the acquisition request in the plurality of different medical departments, all of the electronic medical records 31 created for the patient in the plurality of respective medical departments are acquired. It should be noted that a case is described in which the report 23 is acquired before the electronic medical record 31 is acquired, but the present disclosure is not limited to this. The report 23 may be acquired after the electronic medical record 31 is acquired, or the acquisition of the report 23 and the acquisition of the electronic medical record 31 may be performed in parallel.

In step S3, the list information generation unit 13 records the summary of the information described in the report 23 acquired in step S1 and the electronic medical record 31 acquired in step S2 in the matrix format in which the identification name of the medical department is taken on the first axis (horizontal axis) and the date is taken on the second axis (vertical axis) intersecting the first axis. The list information generation unit 13 specifies, based on the medical department information and the date information assigned to the report 23 and the electronic medical record 31, the recording column of the information described in these medical documents within the format, and records the information corresponding to the specified recording column. As a result, the list information 200 in which the information described in the medical document created for the specific patient in each of the plurality of medical departments is presented in time series for each medical department is generated.

In step S4, the list information generation unit 13 transmits the list information 200 generated in step S3 to the user terminal device 40. A display 41 of the user terminal device 40 that has received the list information 200 displays the list information 200.

Since the information presented in the list information 200 is the summary of the text described in the report 23 or the electronic medical record 31, the user may want to confirm the details of the information described in the report 23 or the electronic medical record 31. The user can, for example, click any cell, in which the summarized information is described, in the list information 200 displayed on the display of the user terminal device 40, to request the details of the information described in the cell to the information processing server 100.

In step S5, the list information generation unit 13 determines whether or not there is the presentation request for the details (detailed information) of the information presented in the list information 200. In a case in which a determination is made that the presentation request for the detailed information is present, the processing proceeds to step S6. On the other hand, in a case in which a determination is made that the presentation request for the detailed information is absent, the present routine ends.

In step S6, the list information generation unit 13 transmits the details (detailed information) of the information described in the report 23 or the electronic medical record 31 in response to the request to the user terminal device 40. The display of the user terminal device 40 displays the detailed information transmitted from the information processing server 100.

As described above, the information processing server 100 according to the present embodiment acquires the medical document (report 23 and electronic medical record 31) to which the medical department information indicating the medical department that is the creation source and the date information indicating the creation date are assigned. The information processing server 100 generates the list information 200 in which the information described in the corresponding medical document is recorded in the format in which the identification name of the medical department is taken on the first axis and the date is taken on the second axis intersecting the first axis. With the information processing server 100 according to the present embodiment, the list information 200 in which the information described in the medical document created for the specific patient in each of the plurality of medical departments is presented in time series for each medical department is generated. As a result, it is possible to understand the information described in the medical documents created for the specific patient in the plurality of medical departments from an overarching perspective.

### Second Embodiment

There is a case in which the doctor belonging to a certain medical department (hereinafter, referred to as a first medical department) requests another medical department (hereinafter, referred to as a second medical department) to handle a finding discovered in the examination or the like. In such a case, the electronic medical record 31 of each of the first medical department and the second medical department may include other-department handling information indicating a matter that needs to be handled by the second medical department. Therefore, the other-department handling information can also be recorded in the list information 200. The information processing server 100 according to the present embodiment presents the other-department handling information in a different form from the other information in the list information 200 in order to prevent the overlooking of the other-department handling information.

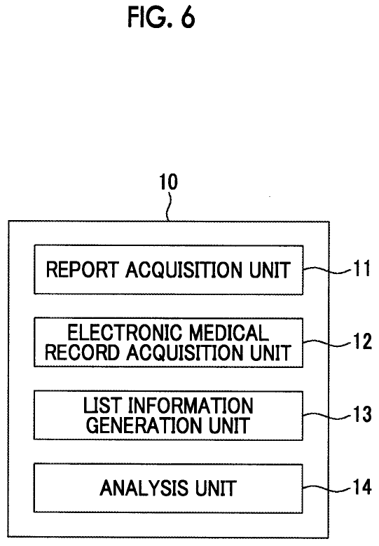

FIG. 6 is a functional block diagram showing an example of a functional configuration of the information processing server 100 according to the second embodiment. The information processing server 100 according to the second embodiment is different from the information processing server 100 according to the first embodiment described above in that an analysis unit 14 is provided.

The analysis unit 14 extracts the other-department handling information described in the electronic medical record 31 by performing the text analysis on the electronic medical record 31 acquired by the electronic medical record acquisition unit 12. A display label indicating that the information is the other-department handling information, such as [handled by other department], is assigned to the other-department handling information described in the electronic medical record 31. The analysis unit 14 extracts the other-department handling information described in the electronic medical record 31 based on the display label. It should be noted that the other-department handling information may be extracted based on a description part indicating a request for handling in the other department in the electronic medical record 31 other than the display label.

The list information generation unit 13 generates the list information including the other-department handling information extracted by the analysis unit 14. In the list information 200 shown in FIG. 4, “chest CT”, which is other-department handling information 201 requested in the internal medicine department, is described in a column corresponding to the internal medicine department, which is a request source, and a column corresponding to the respiratory surgery department, which is a request destination in the format of the list information 200. The list information generation unit 13 presents the other-department handling information 201 in a different form from the other information by performing emphasis display such as highlight display of the other-department handling information 201 in the list information 200. For example, in a case in which the other-department handling information in which the second medical department is referred to is described in a first electronic medical record of the first medical department, the other-department handling display may be performed in both the first medical department and the second medical department. In such a case, the other-department handling information in the column of the first medical department with which the first electronic medical record is associated and other-company handling information in the column of the second medical department with which the first electronic medical record is not associated may be associated with each other in different display aspects. It should be noted that the different display aspects indicate that the cell, the character, the chroma saturation, the lightness, the brightness, the pattern, or the like is changed, the frame, the underline, or the like is added, or the display is performed in a visually identifiable manner. In this way, it is possible to easily understand the medical department that actually describes the first electronic medical record and the medical department described in the first electronic medical record of the other medical department. Further, in a second electronic medical record in which the second medical department is associated after the first electronic medical record, the display aspect of the other-company handling information of the first electronic medical record may be changed in accordance with whether or not a diagnosis regarding the disease described in the first electronic medical record is made. For example, the information processing server 10 stores a corresponding medical department table showing medical department information corresponding to each disease, extracts the disease described in each electronic medical record 31, and determines whether or not the disease extracted based on the corresponding medical department table corresponds to the medical department corresponding to the electronic medical record 31 in which the disease is extracted, thereby displaying the description about the other-department disease corresponding to the medical department other than the medical department corresponding to the electronic medical record 31 in which the disease is extracted, as other-department handling information, in a column of the other department to which the other-department disease is associated in the corresponding medical department table. Then, in a case in which the description about the other-department disease is extracted in the electronic medical record 31 associated with the other department, the display aspect of the other-department handling information is set to a display aspect different from the display aspect before the extraction. In this way, it is possible to determine whether or not the disease discovered in a medical department other than the medical department that handles the disease is appropriately treated in the medical department that handles the disease.

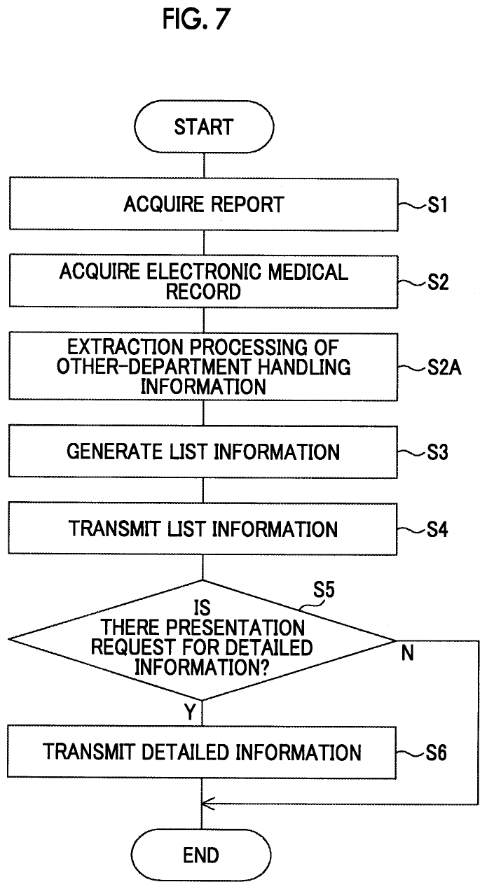

FIG. 7 is a flowchart showing an example of a flow of the processing performed by executing the information processing program 110 via the CPU 101 according to the present embodiment. In the present flowchart, step S2A is added to the flowchart shown in FIG. 5. Hereinafter, only steps different from the flowchart shown in FIG. 5 will be described, and the description of the other steps will be omitted.

In step S2A, the analysis unit 14 performs, for each medical department, the extraction processing of extracting the other-department handling information described in the electronic medical record 31 by performing the text analysis on the electronic medical record 31 acquired in step S2.

In step S3, the list information generation unit 13 generates the list information 200 including the other-department handling information extracted in step S2A. The list information generation unit 13 presents the other-department handling information 201 in a different form from the other information by performing the emphasis display such as the highlight display of the other-department handling information 201 in the list information 200.

With the information processing server 100 according to the present embodiment, since the other-department handling information 201 is presented in a different form from the other information in the list information 200, it is possible to prevent the overlooking of the other-department handling information 201.

### Third Embodiment

In an examination performed in a certain medical department (hereinafter, referred to as a first medical department), a lesion other than a target lesion may be discovered. A finding related to such a lesion is referred to as an incidental finding. The incidental finding discovered in the first medical department is described in the report 23 created in the first medical department, but may not be described in the electronic medical record 31. For the finding that is not described in the electronic medical record 31, there is a risk that an appropriate treatment is not performed and the finding is left as it is. Therefore, the information processing server 100 according to the present embodiment records information on the incidental finding in the list information 200 in order to prevent the incidental finding from being overlooked.

In the information processing server 100 according to the present embodiment, the analysis unit 14 performs, for each medical department, the extraction processing of extracting the finding that is described in the report 23 but is not described in the electronic medical record 31 as the incidental finding. The analysis unit 14 further specifies the medical department that needs to be in charge of the extracted incidental finding. The analysis unit 14 specifies the medical department that needs to be in charge of the incidental finding, for example, based on a part of the report 23 including the description of the incidental finding, that is, a part including the description of an occurrence part or a diagnosis name of the incidental finding. The analysis unit 14 may specify the medical department in charge by using a table in which the occurrence part or the diagnosis name of the incidental finding and the medical department that needs to be in charge of the incidental finding are associated with each other.

The list information generation unit 13 generates the list information 200 including the information on the incidental finding extracted by the analysis unit 14. Specifically, the list information generation unit 13 records the information on the incidental finding, which is extracted by the text analysis of the medical document created in the first medical department, in a column, of the format of the list information 200, corresponding to the first medical department and in a column, of the format of the list information 200, corresponding to the second medical department that needs to be in charge of the incidental finding and that is specified by the analysis unit 14. In the list information 200 shown in FIG. 4, “rib fracture”, which is an incidental finding 202 discovered in the respiratory surgery department, is described in a column, of the format of the list information 200, corresponding to the respiratory surgery department and in a column corresponding to the orthopedic surgery department. The orthopedic surgery department corresponds to the medical department that needs to be in charge of “rib fracture” that is the incidental finding 202.

The list information generation unit 13 presents information (hereinafter, referred to as first incidental finding information) on the incidental finding recorded in the column, of the format, corresponding to the first medical department and information (hereinafter, referred to as second incidental finding information) on the incidental finding recorded in the column, of the format, corresponding to the second medical department, in the list information 200 in a different form from the other information by performing the emphasis display such as the highlight display. The list information generation unit 13 may present the first incidental finding information and the second incidental finding information in different forms from each other. For example, the color of the character or the cell or the background color may be set to be different between the first incidental finding information and the second incidental finding information.

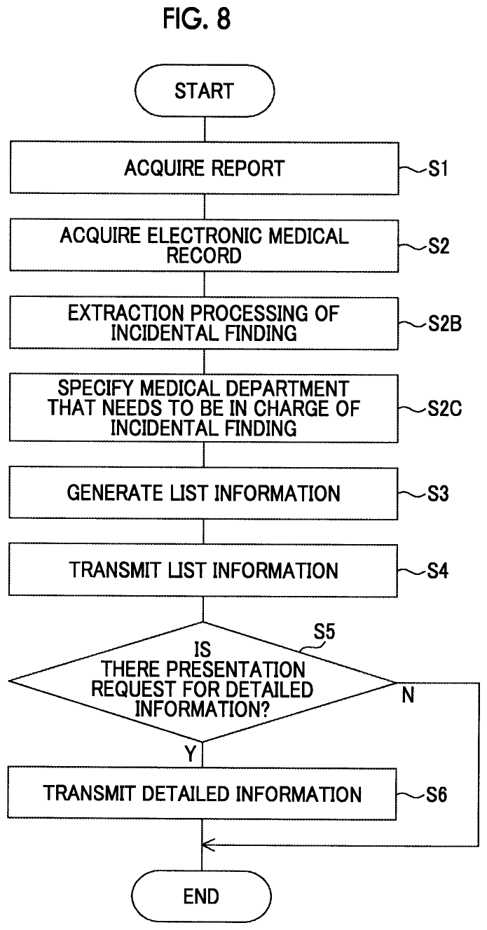

FIG. 8 is a flowchart showing an example of a flow of the processing performed by the CPU 101 according to the present embodiment executing the information processing program 110. In the present flowchart, steps S2B and S2C are added to the flowchart shown in FIG. 5. Hereinafter, only steps different from the flowchart shown in FIG. 5 will be described, and the description of the other steps will be omitted.

In step S2B, the analysis unit 14 performs the text analysis on the report 23 acquired in step S1 and the electronic medical record 31 acquired in step S2 to perform, for each medical department, the extraction processing of extracting the finding that is not described in the electronic medical record 31 among the findings described in the report 23 as the incidental finding.

In step S2C, the analysis unit 14 specifies the medical department that needs to be in charge of the incidental finding among the incidental findings extracted in step S2B.

In step S3, the list information generation unit 13 generates the list information 200 including the information on the incidental finding extracted in step S2B. Specifically, the list information generation unit 13 records the information on the incidental finding, which is extracted by the text analysis of the medical document created in the first medical department, in a column, of the format, corresponding to the first medical department and in a column, of the format, corresponding to the second medical department that needs to be in charge of the incidental finding and that is specified in step S2C. The list information generation unit 13 presents the first incidental finding information on the incidental finding recorded in the column, of the format, corresponding to the first medical department and the second incidental finding information on the incidental finding recorded in the column, of the format, corresponding to the second medical department, in the list information 200 in a different form from the other information by performing the emphasis display such as the highlight display. The list information generation unit 13 presents the first incidental finding information and the second incidental finding information in different forms from each other.

With the information processing server 100 according to the present embodiment, the information on the incidental finding extracted by the text analysis of the medical document created in the first medical department is recorded in the column, of the format of the list information 200, corresponding to the first medical department and the column, of the format of the list information 200, corresponding to the second medical department that needs to be in charge of the incidental finding, and thus it is possible to prevent the incidental finding from being overlooked.

### Fourth Embodiment

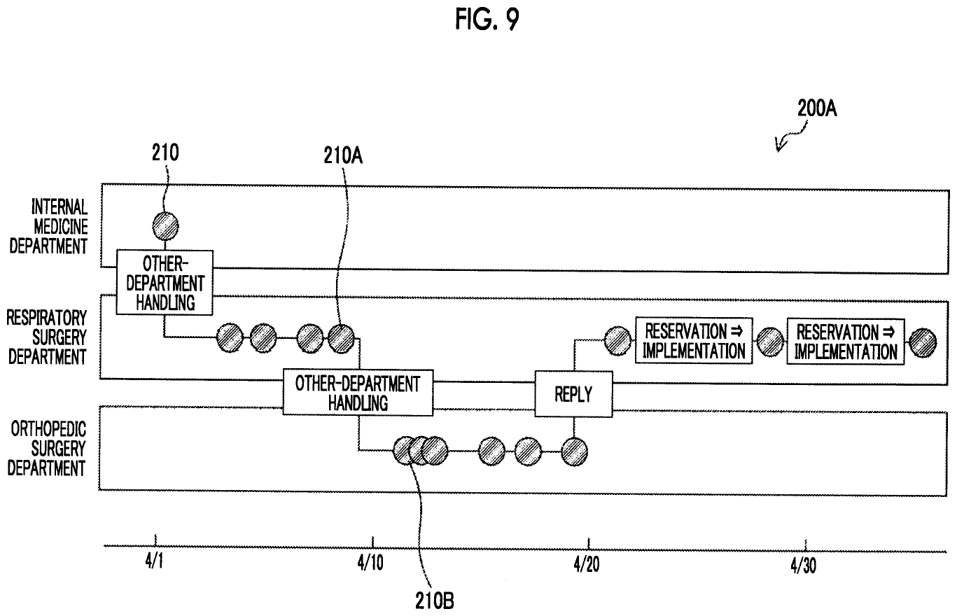

FIG. 9 is a diagram showing an example of list information 200A generated by the information processing server 100 according to the fourth embodiment of the technology of the present disclosure.

The list information generation unit 13 according to the present embodiment generates the list information 200A in which markers 210 corresponding to the report 23 and the electronic medical record 31 are recorded in a matrix format in which the identification name of the medical department is taken on a first axis (vertical axis) and the date is taken on a second axis (horizontal axis) intersecting the first axis.

The list information generation unit 13 specifies, based on the medical department information and the date information each assigned to the report 23 acquired by the report acquisition unit 11 and the electronic medical record 31 acquired by the electronic medical record acquisition unit 12, a recording position of the marker 210 within the format, and records the marker 210 at the specified recording position. As a result, the list information 200A in which the creation or the update of the medical document for the specific patient is presented in time series for each medical department is generated.

In the list information 200A, two markers 210 corresponding to the two medical documents that are created or updated in the different medical departments and that describe the common other-department handling information are connected by a line segment. That is, a marker 210A corresponding to the medical document, which is created in the first medical department and includes the other-department handling information indicating the matter that needs to be handled by the second medical department, and a marker 210B corresponding to the medical document, which is created in the second medical department and includes the other-department handling information that is the same as the above-described other-department handling information, are connected by a line segment.

According to the list information 200A according to the present embodiment, the information on the medical document created in the plurality of medical departments can be confirmed from an overarching perspective. In addition, since the point in time when the medical document is created or updated is indicated by the marker 210, a creation or update interval between the medical documents can be easily understood. It is possible to easily specify the medical document including the same other-department handling information. In addition, it is possible to visualize an aspect of the cooperation among the plurality of medical departments.

It should be noted that, in the list information 200A, the medical document to be recorded with the marker 210 may be limited. For example, in the list information 200A, the marker 210 may be recorded only for the medical document in which the information on the specific disease is described.

In each of the above-described embodiments, for example, as the hardware structure of the processing units that execute various types of processing, such as the report acquisition unit 11, the electronic medical record acquisition unit 12, the list information generation unit 13, and the analysis unit 14, various processors shown below can be used. The various processors include, in addition to a CPU and a GPU which are general-purpose processors that execute software (program) and function as various processing units as described above, a programmable logic device (PLD) which is a processor of which a circuit configuration can be changed after manufacture, such as an FPGA, and a dedicated electric circuit which is a processor of which a circuit configuration is designed for exclusive use in order to execute specific processing, such as an application specific integrated circuit (ASIC).

One processing unit may be configured by one of the various processors or may be configured by combining two or more processors of the same type or different types (for example, by combining a plurality of FPGAs or combining a CPU and an FPGA). A plurality of processing units may be configured by one processor.

A first example of the configuration in which the plurality of processing units are configured by one processor is a form in which one processor is configured by combining one or more CPUs and the software and this processor functions as the plurality of processing units, as represented by computers such as a client and a server. A second example thereof is a form in which a processor that realizes the function of the entire system including the plurality of processing units by one integrated circuit (IC) chip is used, as represented by a system on chip (SoC) or the like. In this way, various processing units are configured by one or more of the various processors as the hardware structure.

Further, the hardware structure of the various processors is, more specifically, an electric circuit (circuitry) in which circuit elements such as semiconductor elements are combined.

Moreover, in the above-described embodiments, an aspect is described in which the information processing program 110 is stored (installed) in advance in the non-volatile memory 103, but the technology of the present disclosure is not limited to this. The information processing program 110 may be provided in a form of being recorded in the recording medium such as a compact disc read only memory (CD-ROM), a digital versatile disc read only memory (DVD-ROM), and a universal serial bus (USB) memory. The information processing program 110 may be provided in a form being downloaded from an external device via a network. That is, the program (that is, a program product) described in the present embodiment may be provided using the recording medium or may be distributed from an external computer.

In regard to the first to fourth embodiments, the following supplementary notes will be further disclosed.

### Supplementary Note 1

An information processing apparatus comprising: at least one processor, in which the processor is configured to: acquire a medical document to which medical department information indicating a medical department that is a creation source and date information indicating a creation date are assigned; and generate list information in which information on a corresponding medical document is recorded in a format in which an identification name of the medical department is taken on a first axis and a date is taken on a second axis intersecting the first axis.

### Supplementary Note 2

The information processing apparatus according to supplementary note 1, in which the information on the medical document is a summary of information described in the medical document.

### Supplementary Note 3

The information processing apparatus according to supplementary note 2, in which the processor is configured to present, in response to a request, details of the information described in the medical document.

### Supplementary Note 4

The information processing apparatus according to any one of supplementary notes 1 to 3, in which the processor is configured to present other-department handling information, which is included in the medical document created by a first medical department and indicates a matter that needs to be handled by a second medical department, in the list information in a different form from other information.

### Supplementary Note 5

The information processing apparatus according to supplementary note 4, in which the processor is configured to extract the other-department handling information via a text analysis of the medical document created by the first medical department.

### Supplementary Note 6

The information processing apparatus according to any one of supplementary notes 1 to 5, in which the processor is configured to record information on an incidental finding extracted by a text analysis of the medical document created by a first medical department in a column, of the format, corresponding to the first medical department and a column, of the format, corresponding to a second medical department that needs to be in charge of the incidental finding.

### Supplementary Note 7

The information processing apparatus according to supplementary note 6, in which the processor is configured to present the information on the incidental finding recorded in the column, of the format, corresponding to the first medical department and the information on the incidental finding recorded in the column, of the format, corresponding to the second medical department, in the list information in different forms from each other.

### Supplementary Note 8

The information processing apparatus according to supplementary note 1, in which the processor is configured to generate the list information in which a marker corresponding to the medical document is recorded in the format.

### Supplementary Note 9

The information processing apparatus according to supplementary note 8, in which the processor is configured to generate the list information in which a first marker corresponding to a medical document, which is created by a first medical department and includes other-department handling information indicating a matter that needs to be handled by a second medical department, and a second marker corresponding to a medical document, which is created by the second medical department and includes information corresponding to the other-department handling information, are connected by a line segment.

### Supplementary Note 10

An information processing method including: via at least one processor provided in an information processing apparatus, acquiring a medical document to which medical department information indicating a medical department that is a creation source and date information indicating a creation date are assigned; and generating list information in which information on a corresponding medical document is recorded in a format in which an identification name of the medical department is taken on a first axis and a date is taken on a second axis intersecting the first axis.

### Supplementary Note 11

An information processing program causing at least one processor of an information processing apparatus to execute a process including: acquiring a medical document to which medical department information indicating a medical department that is a creation source and date information indicating a creation date are assigned; and generating list information in which information on a corresponding medical document is recorded in a format in which an identification name of the medical department is taken on a first axis and a date is taken on a second axis intersecting the first axis.

It should be noted that the disclosure of JP2022-068442 filed on Apr. 18, 2022 is incorporated in the present specification in its entirety by reference. All of the documents, the patent applications, and the technical standards described in the present specification are incorporated herein by reference to the same extent as in a case in which each document, each patent application, and each technical standard are specifically and individually described by being incorporated in the present specification by reference.

What is claimed is:

1. An information processing apparatus comprising:
at least one processor,
wherein the processor is configured to:
acquire a medical document to which medical department information indicating a medical department that is a creation source and date information indicating a creation date are assigned; and
generate list information in which information on a corresponding medical document is recorded in a format in which an identification name of the medical department is taken on a first axis and a date is taken on a second axis intersecting the first axis.

2. The information processing apparatus according to claim 1,
wherein the information on the medical document is a summary of information described in the medical document.

3. The information processing apparatus according to claim 2,
wherein the processor is configured to present, in response to a request, details of the information described in the medical document.

4. The information processing apparatus according to claim 1,
wherein the processor is configured to present other-department handling information, which is included in the medical document created by a first medical department and indicates a matter that needs to be handled by a second medical department, in the list information in a different form from other information.

5. The information processing apparatus according to claim 4,
wherein the processor is configured to extract the other-department handling information via a text analysis of the medical document created by the first medical department.

6. The information processing apparatus according to claim 1,
wherein the processor is configured to record information on an incidental finding extracted by a text analysis of the medical document created by a first medical department in a column, of the format, corresponding to the first medical department and a column, of the format, corresponding to a second medical department that needs to be in charge of the incidental finding.

7. The information processing apparatus according to claim 6,
wherein the processor is configured to present the information on the incidental finding recorded in the column, of the format, corresponding to the first medical department and the information on the incidental finding recorded in the column, of the format, corresponding to the second medical department, in the list information in different forms from each other.

8. The information processing apparatus according to claim 1,
wherein the processor is configured to generate the list information in which a marker corresponding to the medical document is recorded in the format.

9. The information processing apparatus according to claim 8,
wherein the processor is configured to generate the list information in which a first marker corresponding to a medical document, which is created by a first medical department and includes other-department handling information indicating a matter that needs to be handled by a second medical department, and a second marker corresponding to a medical document, which is created by the second medical department and includes information corresponding to the other-department handling information, are connected by a line segment.

10. An information processing method comprising:
via at least one processor provided in an information processing apparatus,
acquiring a medical document to which medical department information indicating a medical department that is a creation source and date information indicating a creation date are assigned; and
generating list information in which information on a corresponding medical document is recorded in a format in which an identification name of the medical department is taken on a first axis and a date is taken on a second axis intersecting the first axis.

11. A non-transitory computer-readable storage medium storing an information processing program causing at least one processor of an information processing apparatus to execute a process comprising:
acquiring a medical document to which medical department information indicating a medical department that is a creation source and date information indicating a creation date are assigned; and
generating list information in which information on a corresponding medical document is recorded in a format in which an identification name of the medical department is taken on a first axis and a date is taken on a second axis intersecting the first axis.
